(19) United States
(12) Patent Application Publication
(10) Pub. No.: US 2011/0092693 A1
(43) Pub. Date: Apr. 21, 2011

(54) NOVEL COMPOUNDS

(75) Inventors: Francois Jean-Charles Natt, Hesingue (FR); Jurg Hunziker, Aarau (CH); Robert Haner, Uettligen (CH); Simon Matthias Langenegger, Wiler (CH)

(73) Assignee: NOVARTIS AG, Basel (CH)

(21) Appl. No.: 12/161,375

(22) PCT Filed: Jan. 16, 2007
(86) PCT No.: PCT/EP2007/000337

§ 371 Date: Jul. 18, 2008

(30) Foreign Application Priority Data
Jan. 18, 2006 (GB) 0601031.8

(51) Int. Cl. C07H 1/00; C07F 9/02

(52) U.S. Cl. 536/25.34; 558/167

(57) ABSTRACT

The present invention relates to a process for the preparation of an oligomeric compound made up of two or more individual oligomers, in which said oligomeric compound the individual oligomers are separated by a photocleavable linker, comprising the step of photoactively cleaving said linker.

### BACKGROUND OF THE INVENTION

Preparation of a double stranded DNA or RNA usually involves two independent multi-step processes (i.e. synthesis, deprotection, purification and quality assurance). While not an issue for most applications, this becomes rate-limiting for scaling up the technology, e.g. for high-throughput applications or for therapeutic applications which require large amount of oligonucleotides. One approach, described by Pon et al [1], termed tandem synthesis, is based on the principle that one (long) oligonucleotide containing a post-synthetically cleavable linker is prepared. Subsequent cleavage then yields the two complementary strands (illustrated in Scheme 1). According to Pon, Richard T.; Yu, Shuyuan. Nucleic Acids Research 2005, 33(6), 1940-1948 and Pon, Richard T.; Yu, Shuyuan. PCT Int. Appl. 2002), WO 2002020537 A2 and Ferreira, Fernando; Meryer, Albert; Vasseur, Jean-Jacques; Morvan, Francois. J. Org. Chem. Online publication, 2005., two or more oligonucleotides separated with a base-labile linker are synthesized sequentially. The linker is then cleaved under the conditions used for the support cleavage and the base/phosphodiester deprotection of the oligonucleotides. One drawback with this procedure is that it does not allow the purification of the oligonucleotides by the trityl-on approach since only the 5′-terminal oligonucleotide will bear this residue. Incorporation of photocleavable residues in oligonucleotides has been described for reversible labeling or immobilization of oligonucleotides and in applications such as SNP genotyping, WO 9967619, or as protecting group in RNA synthesis Stutz, Alfred; Pitsch, Stefan. Synlett 1999, (Spec.), 930-934. Recently, photoactivatable siRNAs or “caged interfering RNAs” have been reported. In these cases, the siRNA antisense strand was modified either on its 5′-end by the introduction of a photocleavable moiety bearing a label group, WO2004045547, or internally by the covalent attachment of 4,5-dimethoxy-2-nitrophenyl groups to the oligoribonucleotide phosphodiester backbone Shah, Samit; Rangarajan, Subhashree; Friedman, Simon, H. Angew. Chem. Int. Ed. 2005, 44, 1328-1332. As such, the oligonucleotide could be photo-activated at a desired time point of the biological experiment, e.g. after its transfection in a cell. The inventors have developed a compound which can be used to simplify the process of synthetically preparing double stranded ribonucleic acids, and provides a method which has several advantages over existing methods. Especially, the use of the compounds of the present invention simplifies the process of the synthetic preparation of double-stranded ribonucleic acids such as siRNAs. By performing the method according to the invention, both strands of a double stranded ribonucleic acid can be obtained from a single synthesis without compromising the quality of the reagent, since it is possible to purify the photocleavable oligonucleotide before release of both strands through irradiation. This feature can be of particular importance in high-throughput applications (e.g. siRNA libraries) or in large scale applications (e.g. siRNA therapeutics). The photocleavable nucleic acids can also be used as such in enzymatic applications (e.g. the incorporation in plasmids), or in biological experiments (e.g. in cellular assay or in animal model assay) and released at any stage of the experiment. Lastly, the inter-oligonucleotide photocleavable linker can be designed to integrate additional functionalities such as label residues or cargo residues which may allow its detection of enhance its pharmacological properties

The inventors have developed a new synthesis strategy using a novel photocleavable linker for the one-step synthesis of multiple compounds. The linker and the use thereof is applicable to the preparation of multiple biopolymers such as for instance polypeptides, polysaccharides or polynucleotides or combinations thereof. It can be especially useful in applications where a controlled ratio of two or more reagents is required. It is particularly suited, but not limited, to the preparation of short interfering RNAs (siRNAs) since it allows the synthesis of both strands as one long self-complementary oligonucleotide with a photocleavable linker resulting after oligonucteotide deprotection and purification in one long oligonucleotide which can also be called photocleavable short hairpin RNA (photo-shRNA).

With respect to siRNA synthesis, this strategy offers the following advantages over standard siRNA preparation; only one molecule is synthesized, purified, and analyzed; light irradiation can be performed on a purified photo-shRNA which consequently ensures the annealing of the siRNA duplex with a perfect stoichiometry; sample tracking of individual strands is not required since non-annealed strands never exist; light irradiation of photo-shRNA to release siRNA can be done at any time, even in biological experiments (e.g. in situ irradiation of photo-shRNA post-transfection or post-injection); and

the linker may be derived to bear functional groups which may enhance cellular uptake or tissue-specific delivery.

The results disclosed herein show that the proposed ortho-nitrobenzyl based linkers are perfectly compatible to standard RNA or DNA oligonucleotide synthesis using phosphoramidite chemistry. The linkers are stable under cleavage and deprotection conditions required to release crude oligonucleotides as well as the aqueous acidic conditions required removing the terminal 5′-dimethoxytrityl group. The present invention provides a compound and the use of the compound which allows the synthesis of multiple purified oligonucleotides in a single synthesis process. In its current form, cleavage of the linker by light irradiation releases oligonucleotides bearing a terminal phosphate residue at the linker anchoring terminus. While this may be a disadvantage for some applications requiring terminal hydroxyl groups, it turns to be an advantage for the preparation of siRNAs which require a phosphate group at the 5′-terminus of the guide strand for biological function Meister, Gunter; Tuschl, Thomas. Nature 2004, 431(7006), 343-349.

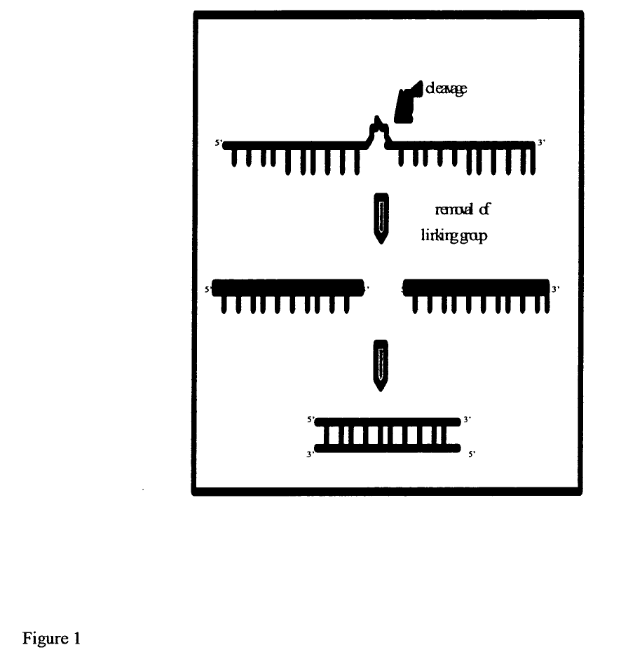

The simplicity of the method according to the invention is shown in FIG. 1.

In a first aspect the invention relates to a process for the preparation of an oligomeric compound made up of two or more individual oligomers, in which said oligomeric compound the individual oligomers are separated by a photocleavable linker, comprising the step of photoactively cleaving said linker.

The individual oligomers may be independently chosen from the group consisting of oligonucleotides, oligosaccharides, oligopeptides.

In one embodiment, the individual oligomers are oligonucleotides which may or may not be complementary. Preferably, the oligomers are fully or partially complementary. Partial complementarity means that 50%-99% of the nucleotides in the oligonucleotides are complementary.

In a preferred embodiment, the individual oligomers are oligoribonucleotides which may be fully or partially complementary.

In a preferred embodiment, the linker is stable under the deprotection conditions of each individual oligomer.

Preferably, the linker group is cleavable by UV or visible light irradiation.

In a preferred embodiment, said oligonucleotides are two oligoribonucleotides

In an additional embodiment, the linker is a compound of formula I,

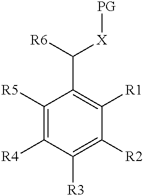

wherein;
PG is (Ar1)(Ar2)(Ar3)C—, wherein Ar1, Ar2, Ar3 are independently chosen from the group consisting of;

### CH₃OC₆H₄— and C₆H₅—,

or PG is a substituted silyl group (R1′)(R2′)(R3′)Si—,
wherein R1′, R2′, R3′ is independently chosen from the group consisting of lower alkyl, aryl, aryl lower alkyl, lower alkylaryl, lower alkyloxy, or aryloxy;

### X is O, N, or S;

R1, R2, R3, R4, and R5 is independently chosen from the group consisting of hydrogen, lower alkyl, aryl, aryl lower alkyl, lower alkylaryl, lower alkylhalogen, halogen, CN, COOH, C(O)O lower alkyl/aryl, CONR′R″, CHO, C(O) lower alkyl/aryl, OH, O-lower alkyl/aryl, OC(O) lower alkyl/aryl, SH, S-lower alkyl/aryl, SO₃H, SO₂O-lower alkyl/aryl, SO₂NR′R″, NH₂, N-lower alkyl/aryl, NHC(O) lower alkyl/aryl, and at least one of the substituents R1-R5 is a nitro, a nitrosyl, or a diazo group;
two or more of the substituents R1, R2, R3, R4, and R5 can form one or several rings which may be further substituted with groups defined as for R1, R2, R3, R4, or R5;
at least one of the substituents R1, R2, R3, R4, or R5 is a phosphoramidite, a phosphonate, or a phosphotriester bearing group able to form a phosphodiester or phosphorothioate linkage to the growing oligonucleotide chain or an amine, an activated carboxylic ester, an isocyanate or an isothiocyanate, able to form an amide, a urea or a thiourea linkage to the growing oligonucleotide chain;
R6 is chosen from the group consisting of hydrogen, lower alkyl, aryl, aryl lower alkyl, lower alkylaryl, lower alkylhalogen, CN, COOH, C(O)O lower alkyl/aryl, CONR′R″, CHO, C(O) lower alkyl/aryl, O-lower alkyl/aryl, OC(O) lower alkyl/aryl, S-lower alkyl/aryl, SO₃H, SO₂O-lower alkyl/aryl, SO₂NR′R″, N-lower alkyl/aryl, NHC(O) lower alkyl/aryl.

This linker is preferably cleavable by light, such as UV light or visible light, or a laser beam.

Even more preferred is a process as described above, wherein the linker is a compound of formula II,

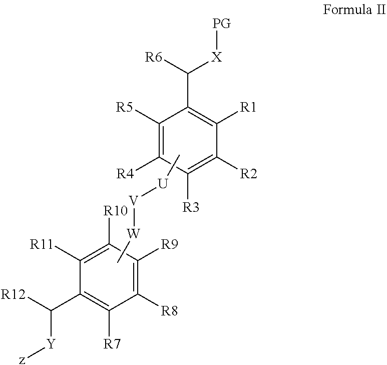

wherein,
PG is (Ar1)(Ar2)(Ar3)C—, wherein Ar1, Ar2, Ar3 are independently chosen from the group consisting of;

### CH₃OC₆H₄—, C₆H₅—,

or PG is a substituted silyl group (R1′)(R2′)(R3′)Si—,
wherein R1′, R2′, R3′ is independently chosen from the group consisting of lower alkyl, aryl, aryl lower alkyl, lower alkylaryl, lower alkyloxy, or aryloxy;

### X is O, N, or S;

R1, R2, R3, R4, and R5 is independently chosen from the group consisting of hydrogen, lower alkyl, aryl, aryl lower alkyl, lower alkylaryl, lower alkylhalogen, halogen, CN, COOH, C(O)O lower alkyl/aryl, CONR′R″, CHO, C(O) lower alkyl/aryl, OH, O-lower alkyl/aryl, OC(O) lower alkyl/aryl, SH, S-lower alkyl/aryl, SO₃H, SO₂O-lower alkyl/aryl, SO₂NR′R″, NH₂, N-lower alkyl/aryl, NHC(O) lower alkyl/aryl, and at least one of the substituents R1-R5 is a nitro, a nitrosyl, or a diazo group;
two or more of the substituents R1, R2, R3, R4, and R5 can form one or several rings which may be further substituted with groups defined as for R1, R2, R3, R4, or R5;
R6 is chosen from the group consisting of hydrogen, lower alkyl, aryl, aryl lower alkyl, lower alkylaryl, lower alkylhalogen, CN, COOH, C(O)O lower alkyl/aryl, CONR′R″, CHO, C(O) lower alkyl/aryl, O-lower alkyl/aryl, OC(O) lower alkyl/aryl, S-lower alkyl/aryl, SO₃H, SO₂O-lower alkyl/aryl, SO₂NR′R″, N-lower alkyl/aryl, NHC(O) lower alkyl/aryl;
U, V, W are forming a chain which replaces one of the substituents R1-R5 on one end and one of the substituents R7-R11 on the other end;
U, V, W can independently be absent, or be an alkylene (—R—), cycloalkylene (—R—), or arylene (—Ar—) group, —O—, —S—, —NR′—, —C(O)—, —C(O)O—, —C(O)NR′—, —OC(O)O—, —OC(O)NR′—, —NR′C(O)NR″—, —OC(S)NR′—, —NR′C(S)NR″—, —S(O)—, —S(O₂)—, —S(O₂)NR′—, —OP(O₂)O—, and may contain a label or fluorophore or a group which serves to improve the pharmacological profile of the oligonucleotide.
R7, R8, R9, R10, and R11 are independently chosen form the group consisting of, hydrogen, lower alkyl, aryl, aryl lower alkyl, lower alkylaryl, lower alkylhalogen, halogen, CN, COOH, C(O)O lower alkyl/aryl, CONR′R″, CHO, C(O) lower alkyl/aryl, OH, O-lower alkyl/aryl, OC(O) lower alkyl/aryl, SH, S-lower alkyl/aryl, SO₃H, SO₂O lower alkyl/aryl, SO₂NR′R″, NH2, N-lower alkyl/aryl, NHC(O) lower alkyl/aryl and at least one of the substituents R7-R11 is a nitro, a nitrosyl, or a diazo group;
R12 is chosen from the group consisting of hydrogen, lower alkyl, aryl, aryl lower alkyl, lower alkylaryl, lower alkylhalogen, CN, COOH, C(O)O lower alkyl/aryl, CONR′R″, CHO, C(O) lower alkyl/aryl, O-lower alkyl/aryl, OC(O) lower alkyl/aryl, S-lower alkyl/aryl, SO₃H, SO₂O-lower alkyl/aryl, SO₂NR′R″, N-lower alkyl/aryl, NHC(O) lower alkyl/aryl;

### Y is O, N, or S;

Z is a phosphoramidite, a phosphonate, or a phosphotriester group able to form a phosphodiester or phosphorothioate linkage to the growing oligonucleotide chain or an amine, an activated carboxylic ester, an isocyanate or an isothiocyanate which is able to form an amide, a urea or a thiourea linkage to the growing oligonucleotide chain.

Even more preferred is a process according to the above, wherein the linker is a compound of formula I,

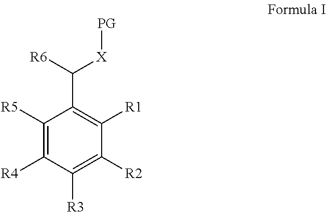

wherein;
PG is dimethoxytriphenylmethyl;

### X is O;

R1 is a nitro group;
R3 is —CH₂—O—P(N[iPr]₂)—O—CH₂—CH₂—CN);
R2, R4, R5, and R6 are hydrogen;

More preferred is a process according to the above, wherein the linker is a compound of formula II

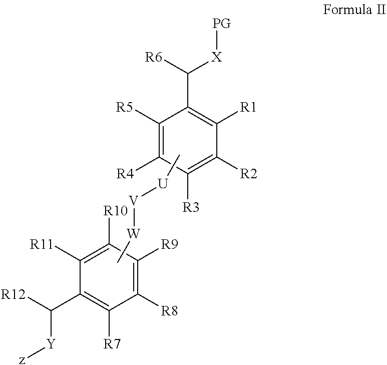

wherein;
PG is dimethoxytriphenylmethyl;

### X and Y are O;

R1 and R7 are nitro groups;
R2, R4, R5, R6, R8, R10, R11, and R12 are hydrogen;
U is oxygen and replaces R3;

### V is —CH₂—CH₂—CH₂—;

W is oxygen and replaces R9;
Z is —P(N[iPr]₂)—O—CH₂—CH₂—CN).

In yet a further embodiment, the present invention provides a compound according to formula I,

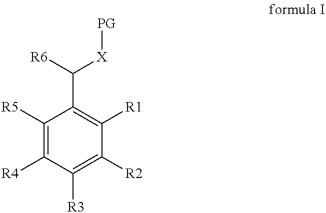

wherein;
PG is (Ar1)(Ar2)(Ar3)C—, wherein Art Ar2, Ar3 are independently chosen from the group consisting of;

### CH₃OC₆H₄— and C₆H₅—,

or PG is a substituted silyl group (R1′)(R2′)(R3′)Si—,
wherein R1, R2′, R3′ is independently chosen from the group consisting of lower alkyl, aryl, aryl lower alkyl, lower alkylaryl, lower alkyloxy, and aryloxy;

### X is O, N, or S;

R1, R2, R3, R4, and R5 is independently chosen from the group consisting of hydrogen, lower alkyl, aryl, aryl lower alkyl, lower alkylaryl, lower alkylhalogen, halogen, CN, COOH, C(O)O lower alkyl/aryl, CONR′R″, CHO, C(O) lower alkyl/aryl, OH, O-lower alkyl/aryl, OC(O) lower alkyl/aryl, SH, S-lower alkyl/aryl, SO₃H, SO₂O-lower alkyl/aryl, SO₂NR′R″, NH₂, N-lower alkyl/aryl, and NHC(O) lower alkyl/aryl; and at least one of the substituents R1-R5 is a nitro, a nitrosyl, or a diazo group;
two or more of the substituents R1, R2, R3, R4, and R5 can form one or several rings which may be further substituted with groups defined as for R1, R2, R3, R4, or R5;
at least one of the substituents R1, R2, R3, R4, or R5 is a phosphoramidite, a phosphonate, or a phosphotriester bearing group able to form a phosphodiester or phosphorothioate linkage to the growing oligonucleotide chain or an amine, an activated carboxylic ester, an isocyanate or an isothiocyanate, able to form an amide, a urea or a thiourea linkage to the growing oligonucleotide chain;
R6 is chosen from the group consisting of hydrogen, lower alkyl, aryl, aryl lower alkyl, lower alkylaryl, lower alkylhalogen, CN, COOH, C(O)O lower alkyl/aryl, CONR′R″, CHO, C(O) lower alkyl/aryl, O-lower alkyl/aryl, OC(O) lower alkyl/aryl, S-lower alkyl/aryl, SO₃H, SO₂O-lower alkyl/aryl, SO₂NR′R″, N-lower alkyl/aryl, and NHC(O) lower alkyl/aryl.

More preferred is a compound of formula II

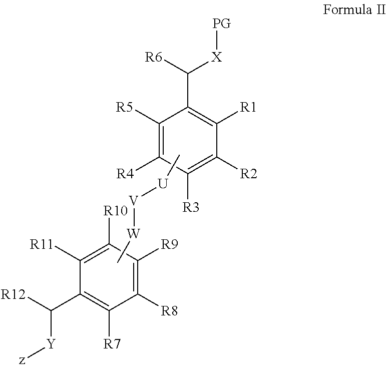

wherein,
PG is (Ar1)(Ar2)(Ar3)C—, wherein Ar1, Ar2, Ar3 are independently chosen from the group consisting of;

### CH₃OC₆H₄—, C₆H₅—,

or PG is a substituted silyl group (R1′)(R2′)(R3′)Si—,
wherein R1′, R2′, R3′ is independently chosen from the group consisting of lower alkyl, aryl, aryl lower alkyl, lower alkylaryl, lower alkyloxy, or aryloxy;

### X is O, N, or S;

R1, R2, R3, R4, and R5 is independently chosen from the group consisting of hydrogen, lower alkyl, aryl, aryl lower alkyl, lower alkylaryl, lower alkylhalogen, halogen, CN, COOH, C(O)O lower alkyl/aryl, CONR′R″, CHO, C(O) lower alkyl/aryl, OH, O-lower alkyl/aryl, OC(O) lower alkyl/aryl, SH, S-lower alkyl/aryl, SO₃H, SO₂O-lower alkyl/aryl, SO₂NR′R″, NH₂, N-lower alkyl/aryl, and NHC(O) lower alkyl/aryl, and at least one of the substituents R1-R5 is a nitro, a nitrosyl, or a diazo group;
two or more of the substituents R1, R2, R3, R4, and R5 can form one or several rings which may be further substituted with groups defined as for R1, R2, R3, R4, or R5;
R6 is chosen from the group consisting of hydrogen, lower alkyl, aryl, aryl lower alkyl, lower alkylaryl, lower alkylhalogen, CN, COOH, C(O)O lower alkyl/aryl, CONR′R″, CHO, C(O) lower alkyl/aryl, O-lower alkyl/aryl, OC(O) lower alkyl/aryl, S-lower alkyl/aryl, SO₃H, SO₂O-lower alkyl/aryl, SO₂NR′R″, N-lower alkyl/aryl, and NHC(O) lower alkyl/aryl;
U, V, W are forming a chain which replaces one of the substituents R1-R5 on one end and one of the substituents R7-R11 on the other end;
U, V, W can independently be absent, or be an alkylene (—R—), cycloalkylene (—R—), or arylene (—Ar—) group, —O—, —S—, —NR′—, —C(O)—, —C(O)O—, —C(O)NR′—, —OC(O)O—, —OC(O)NR′—, —NR′C(O)NR″—, —OC(S)NR′—, —NR′C(S)NR″—, —S(O)—, —S(O₂)—, —S(O₂)NR′—, —OP(O₂)O—, and may contain a label or fluorophore or a group which serves to improve the pharmacological profile of the oligonucleotide.
R7, R8, R9, R10, and R11 are independently chosen form the group consisting of, hydrogen, lower alkyl, aryl, aryl lower alkyl, lower alkylaryl, lower alkylhalogen, halogen, CN, COOH, C(O)O lower alkyl/aryl, CONR′R″, CHO, C(O) lower alkyl/aryl, OH, O-lower alkyl/aryl, OC(O) lower alkyl/aryl, SH, S-lower alkyl/aryl, SO₃H, SO₂O lower alkyl/aryl, SO₂NR′R″, NH₂, N-lower alkyl/aryl, NHC(O) lower alkyl/aryl and at least one of the substituents R7-R11 is a nitro, a nitrosyl, or a diazo group;
R12 is chosen from the group consisting of hydrogen, lower alkyl, aryl, aryl lower alkyl, lower alkylaryl, lower alkylhalogen, CN, COOH, C(O)O lower alkyl/aryl, CONR′R″, CHO, C(O) lower alkyl/aryl, O-lower alkyl/aryl, OC(O) lower alkyl/aryl, S-lower alkyl/aryl, SO₃H, SO₂O-lower alkyl/aryl, SO₂NR′R″, N-lower alkyl/aryl, NHC(O) lower alkyl/aryl;

### Y is O, N, or S;

Z is a phosphoramidite, a phosphonate, or a phosphotriester group able to form a phosphodiester or phosphorothioate linkage to the growing oligonucleotide chain or an amine, an activated carboxylic ester, an isocyanate or an isothiocyanate which is able to form an amide, a urea or a thiourea linkage to the growing oligonucleotide chain.

Even more preferred is a compound of formula I,

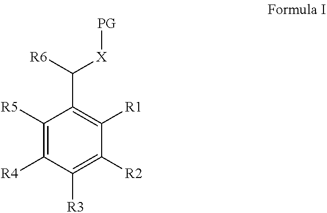

wherein;
PG is dimethoxytriphenylmethyl;

### X is O;

R1 is a nitro group;
R3 is —CH₂—O—P(N[iPr]₂)—O—CH₂—CH₂—CN);
R2, R4, R5, and R6 are hydrogen;

More preferred is a compound according to formula II

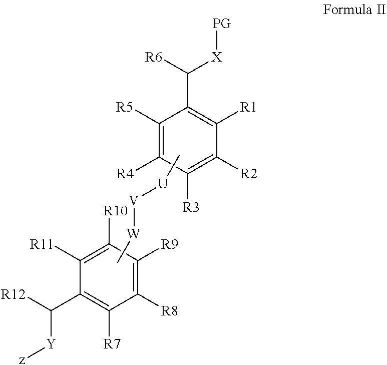

wherein;
PG is dimethoxytriphenylmethyl;

### X and Y are O;

R1 and R7 are nitro groups;
R2, R4, R5, R6, R8, R10, R11, and R12 are hydrogen;
U is oxygen and replaces R3;

### V is —CH₂—CH₂—CH₂—;

W is oxygen and replaces R9;
Z is —P(N[iPr]₂)—O—CH₂—CH₂—CN).

The term “lower” in connection with organic radicals or compounds means a compound or radical which may be branched or unbranched with up to and including 8 carbon atoms, preferably 1-6 or more preferably 1-4, or 2-6 carbon atoms. Lower alkyl represents, for example, methyl, ethyl, n-propyl, isopropyl, n-butyl, sec-butyl, tert-butyl, n-pentyl and branched pentyl, n-hexyl and branched hexyl, n-heptyl, branched heptyl, n-octyl and branched octyl.

iPr means isopropyl.

### Materials and Methods

### Synthesis of Photo-Cleavable Phosphoramidites

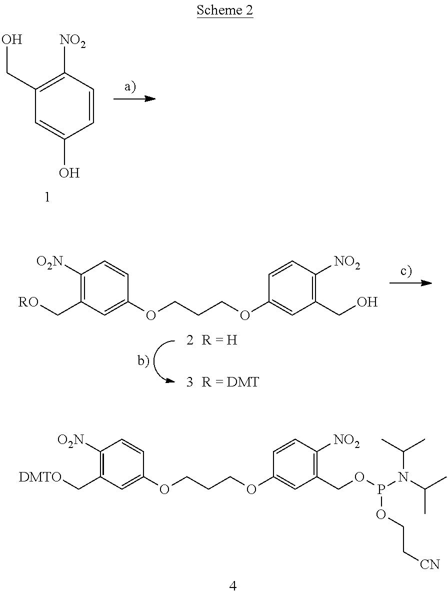

### 3-Hydroxymethyl-4-nitro-phenol (1)

Compound I was synthesized according to the literature R. Reinhard, B. F. Schmidt, J. Org. Chem., 1998, 63, 2434-2441.

### {5-[3-(3-Hydroxymethyl-4-nitro-phenoxy)-propoxy]-2-nitro-phenyl}-methanol (2)

Compound I (2.02 g, 12 mmol) was dissolved in DMF (26 ml). 1,3-Dibromopropane (560 μl, 5.4 mmol), K₂CO₃ (2.0 g, 14.4 mmol) and potassium iodide (0.2 g, 1.2 mmol) were added and the orange suspension was stirred at 90° C. for 3 h. The reaction solution was then cooled to room temperature and poured into 140 ml of water. The precipitate was filtered off, washed with water, sat. aq. NaHCO₃ solution and then again twice with water, dried to give 1.82 g of slightly yellow crystals. Yield 89%. TLC (AcOEt/hexane 1:1): R_{f} 0.21. ¹H-NMR (300 MHz, DMSO-d₆): 2.27 (q, J=6.2, CH₂CH₂CH₂); 4.30 (t, J=6.2, CH₂CH₂CH₂); 4.84 (s, CH₂OH, CH₂OH); 5.59 (s, CH₂OH, C′H₂OH); 7.05 (dd, J=9.1, 2.8, 2 arom. H); 7.36 (d, J=2.8, 2 arom. H); 8.12 (d, J=9.1, 2 arom. H). ¹³C-NMR (75 MHz, DMSO-d₆): 28.2; 60.3; 65.0; 112.8; 113.2; 127.5; 139.4; 142.4; 162.9. HR-ESI-MS (pos. mode): 401.0959 ([M+Na]⁺; calc. 401.0960).

### [5-(3-{3-[Bis-(4-methoxy-phenyl)-phenyl-methoxymethyl]-4-nitro-phenoxy}-propoxy)-2-nitro-phenyl]-methanol (3)

1.8 g (4.76 mmol) 2 was dissolved in 45 ml pyridine under nitrogen. A solution of 1.61 g (4.76 mmol) DMTCl in 20 ml dry pyridine was added at room temperature. The reaction mixture was stirred over night, diluted with sat. aq. NaHCO₃ solution and extracted twice with AcOEt. The combined organic phases were washed with water and Brine, dried (K₂CO₃) and evaporated under reduced pressure. The resulting oil was purified by column chromatography (silicagel; AcOEt/hexane 1:3, 2% Et₃N→AcOEt, 2% Et₃N) to give 1.45 g 3 as a yellow foam. Yield 45%. TLC (AcOEt/hexane 1:1): R_{f} 0.43. ¹H-NMR (300 MHz, CDCl₃): 2.40 (q, J=6.0, CH₂CH₂CH₂); 2.56 (t, J=6.4, CH₂OH); 3.78 (s, 2OMe); 4.33 (t, J=6.0, CH₂CH₂CH₂); 4.65 (s, CH₂ODMT); 4.99 (d, J=6.4, CH₂OH); 6.8-6.95 (m, 6 arom. H); 7.2-7.4 (m, 8 arom. H); 7.47 (m, 2 arom. H); 7.70 (m, 2 arom. H); 8.12 (d, J=9.1, 1 arom. H); 8.18 (d, J=9.1, 1 arom. H). HR-ESI-MS (pos. mode): 703.2264 ([M+Na]⁺; calc. 703.2267).

### Diisopropyl-phosphoramidous acid 5-(3-{3-[bis-(4-methoxy-phenyl)-phenyl-methoxymethyl]-4-nitro-phenoxy}-propoxy)-2-nitro-benzyl ester 2-cyano-ethyl ester (4)

1.0 g (1.47 mmol) 3 was dissolved in 6 ml CH₂Cl₂ under nitrogen. Then 0.6 ml Hünig's base, and 0.38 g (1.62 mmol) 2-cyanoethyl diisopropylamidochlorido-phosphite were added and the mixture was stirred for 3 h at room temperature. The reaction mixture was directly applied onto silica gel and purified by column chromatography (silica gel (50 g); AcOEt/hexane 3:7, 2% Et₃N→AcOEt, 2% Et₃N). 1.05 g 4 as a yellow foam was obtained. Yield 81%. TLC (AcOEt/hexane 1:1): R_{f} 0.79. ¹H-NMR (300 MHz, CDCl₃): 1.21 (d, J=6.9, 2 MeCHN); 2.40 (q, J=6.0, CH₂CH₂CH₂); 2.60 (t, J=6.3, CH₂CN); 3.6-4.0 (m, OCH₂CH₂CN, 2 Me₂CHN); 3.78 (s, 2OMe); 4.32 (t, J=6.0, CH₂CH₂CH₂); 4.65 (s, CH₂ODMT); 5.14 (m, CH₂OP); 6.8-6.95 (m, 6 arom. H); 7.2-7.4 (m, 8 arom. H); 7.47 (m, 2 arom. H); 7.70 (m, 2 arom. H); 8.11 (d, J=9.0, 1 arom. H); 8.18 (d, J=9.1, 1 arom. H). ³¹P-NMR (162 MHz, CDCl₃): 149.12. HR-ESI-MS (pos. mode): 903.3326 ([M+Na]⁺; calc. 903.3346).

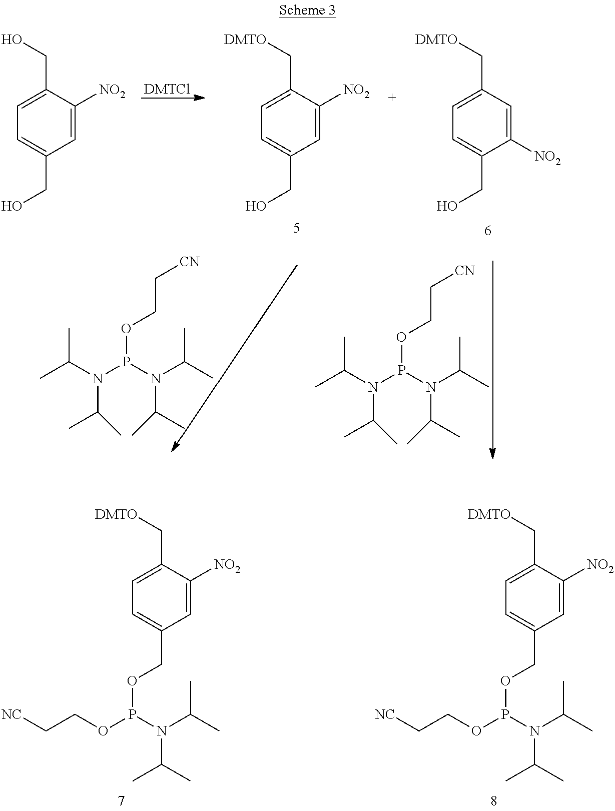

### {4-[Bis-(4-methoxy-phenyl)-phenyl-methoxymethyl]-3-nitro-phenyl}-methanol (5) and {4-[Bis-(4-methoxy-phenyl)-phenyl-methoxymethyl]-2-nitro-phenyl}-methanol (6)

(4-Hydroxymethyl-2-nitro-phenyl)-methanol (TCI Tokyo Kasei, 3.0 g, 16.4 mmol) was dissolved in pyridine (30 ml) under an argon atmosphere. 4,4′-Dimethoxytrityl chloride (5.55 g, 16.4 mmol) was added in portions over a period of 30 minutes while cooling the solution to 0° C. The reaction mixture was stirred over night at room temperature, diluted with sat. aq. NaHCO₃ solution and extracted twice with AcOEt. The combined organic phases were washed with water and brine, dried (NaHCO₃) and evaporated under reduced pressure. The resulting oil was purified by column chromatography (silicagel; AcOEt/hexane 1:4, 1% Et₃N→AcOEt, 1% Et₃N) to give 0.91 g of 5 (11%) and 3.18 g of 6 (40%) as yellow foams.

Analytical data for 5: TLC (AcOEt/hexane 1:2): R_{f} 0.11. ¹H-NMR (300 MHz, CDCl₃): 1.90 (t, J=6.4, CH₂OH); 3.70 (s, 2OMe); 4.52 (s, CH₂ODMT); 4.68 (s, CH₂OH); 6.72-6.79 (m, 4 arom. H); 7.06-8.03 (m, 12 arom. H). EI-MS: 485 [M+•].

Analytical data for 6: TLC (AcOEt/hexane 1:2): R_{f} 0.24. ¹H-NMR (300 MHz, CDCl₃): 1.71 (t, J=6.4, CH₂OH); 3.71 (s, 2OMe); 4.19 (s, CH₂ODMT); 4.85 (s, CH₂OH); 6.73-6.78 (m, 4 arom. H); 7.06-8.03 (m, 12 arom. H). EI-MS: 485 [M+•].

### Diisopropyl-phosphoramidous acid 4-[bis-(4-methoxy-phenyl)-phenyl-methoxymethyl]-3-nitro-benzyl ester 2-cyano-ethyl ester (7)

Alcohol 5 (300 mg, 0.62 mmol) was dissolved in 2.4 ml CH₂Cl₂ under an argon atmosphere. 2-Cyanoethyl-2(diisopropylamido)phosphite (0.28 ml, 0.77 mmol) and tetrazolide (145 mg, 0.846 mmol), dissolved in CH₂Cl₂ (2.4 ml) were added. The mixture was stirred at room temperature for 3 h, diluted with sat. aq. NaHCO₃ solution and extracted twice with CH₂Cl₂. The combined organic phases were dried (NaHCO₃) and concentrated under reduced pressure. The resulting oil was purified by column chromatography (silicagel; AcOEt/hexane 1:4, 1% N-methyl-morpholine) to give 7 (253 mg, 61%) as a yellow foam. TLC (AcOEt/hexane 1:2): R_{f} 0.50. ¹H-NMR (300 MHz, CDCl₃): 1.20 (2d, J=6.8, 4MeCHN); 2.65 (t, J=6.4, CH₂CN); 3.61-3.69 (m, OCH₂CH₂CN); 3.77 (s, OMe); 3.79-3.91 (m, 2Me₂CHN); 4.57 (s, CH₂ODMT); 4.76 (m, CH₂OP); 6.78-6.84 (m, 4 arom. H); 7.20-7.48 (m, 10 arom. H) 7.64 (d, J=8.1, 1 arom. H); 8.01 (s, 1 arom. H); 8.09 (d, J=8.1, 1 arom. H). ³¹P-NMR (162 MHz, CDCl₃): 150.84. ESI-MS (pos. mode): 708 ([M+Na]⁺; calc. 708).

### Diisopropyl-phosphoramidous acid 4-[bis-(4-methoxy-phenyl)-phenyl-methoxymethyl]-2-nitro-benzyl ester 2-cyano-ethyl ester (8)

Alcohol 6 (300 mg, 0.62 mmol) was dissolved in CH₂Cl₂ (2.4 ml) under an argon atmosphere. 2-Cyanoethyl-2(diisopropylamido)phosphite (0.28 ml, 0.77 mmol) and tetrazolide (145 mg, 0.85 mmol), dissolved in CH₂Cl₂ (2.4 ml) were added. The mixture was stirred at room temperature for 3 h, diluted with sat. aq. NaHCO₃ solution and extracted twice with CH₂Cl₂. The combined organic phases were dried (NaHCO₃) and concentrated under reduced pressure. The resulting oil was purified by column chromatography (silicagel; AcOEt/hexane 1:4, 1% N-methyl-morpholine) to give 8 (352 mg, 85%) as a yellow foam. TLC (AcOEt/hexane 1:2): R_{f} 0.42. ¹H-NMR (300 MHz, CDCl₃): 1.14 (2d, J=6.8, 4 MeCHN); 2.58 (t, J=6.4, CH₂CN); 3.54-3.66 (m, OCH₂CH₂CN); 3.72 (s, OMe); 3.75-3.96 (m, 2Me₂CHN); 4.18 (s, CH₂ODMT); 5.00 (m, CH₂OP); 6.75-6.80 (m, 4 arom. H); 7.12-7.42 (m, 10 arom. H); 7.58 (d, J=8.1, 1 arom. H); 7.71 (d, J=8.1, 1 arom. H); 7.97 (s, 1 arom. H). ³¹P-NMR (162 MHz, CDCl₃): 150.35. ESI-MS (pos. mode): 708 ([M+Na]⁺; calc. 708).

### Synthesis of Oligonucleotides.

Oligodeoxynucleotides were synthesized on a 392 DNA/RNA Synthesizer (Applied Biosystems) according to the phosphoramidite chemistry[6,7]. The deoxynucleoside phosphoramidites were from Transgenomic (Glasgow, UK). Oligodeoxynucleotides were prepared by the standard synthetic procedure (“trityl-off” mode). Detachment from the solid support and final deprotection was achieved by treatment with 30% ammonium hydroxide overnight at 55° C.

Oligoribonucleotides were synthesized on a Mermade DNA plate synthesizer (Bioautomation Inc.) according to the TOM protected RNA phosphoramidite chemistry [3]. The ribonucleoside phosphoramidites were from Qiagen AG (Hombrechtikon, CH). Oligonucleotides were prepared according to the standard synthetic procedure (“trityl-on” mode). Detachment from the solid support and base/phosphodiester backbone deprotection was achieved by treatment with aqueous Ammonia/Methylamine solution (1:1) for 30 minutes at 65° C. 2′-TOM deprotection was achieved by treatment with TEA-HF solution for 1 h at 65° C.

### Purification of Oligonucleotides

Where specified, oligonucleotides were purified with OASIS cartridges (Waters AG). First, the cartridge was conditioned with 1 ml acetonitrile followed by 1 ml of 0.1M of triethylammonium acetate solution (TEAA). The crude oligonucleotides was loaded on the cartridge which was washed with a 15% acetonitrile solution in 0.1M TEAA to remove all trityl-off truncated sequences. On-cartridge detritylation was performed with 1 ml of an aqueous 3% dichloroacetic acid solution. Before elution of the purified trityl-off oligonucleotide with a 1:1 acetonitrile/water solution, the cartridge was washed with 1-2 ml of 0.1M TEAA or water.

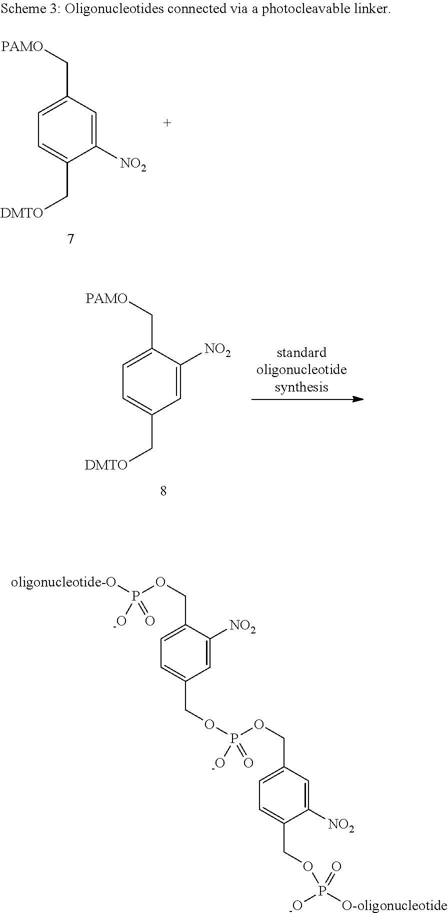

### Photocleavage of Oligonucleotides.

Cleavage of oligonucleotides was performed by irradiation of a solution of the oligonucleotide (0.1 to 10 optical densities) in water (10-100 microliters in a conventional plastic cuvette) with light (352 nm wavelength; two 8 Watt tubes) for 15 to 180 minutes. The treatment resulted in the formation of two individual oligonucleotides (Scheme 4 and FIG. 1).

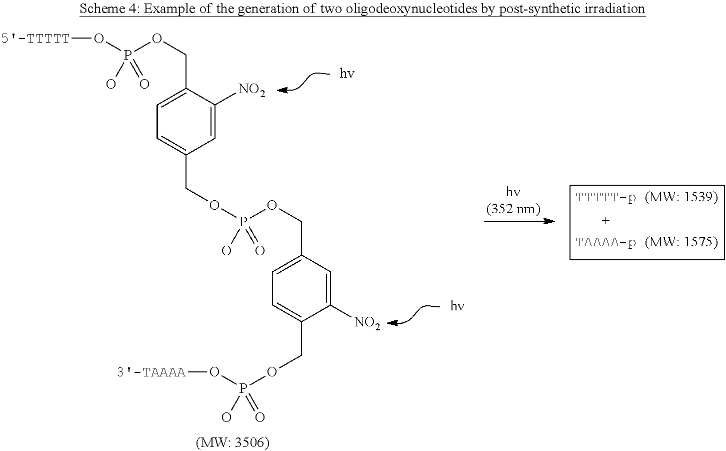

TABLE 1
MS analysis of oligonucleotides before and after irradiation.

| | Mcalc. | Mmeas. |
|---|---|---|
| Before irradiation | 3506 | 3506.00 |
| After irradiation | 1575 | 1574.63 |
| | 1539 | 1538.63 |

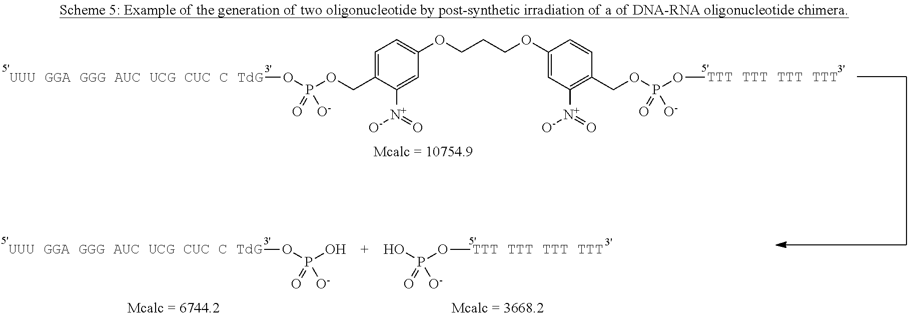

TABLE 2
MS analysis of oligonucleotides before and after irradiation.

| | Mcalc. | Mmeas. |
|---|---|---|
| Before irradiation | 10754.9 | 10757.87 |
| After irradiation | 6744.2 | 6745.56 |
| | 3668.2 | 3668.82 |

A first photocleavable oligodeoxynucleotide was prepared using standard phosphoramidite chemistry by concomitant incorporation of phosphoramidites 8 and 7 on the 5′ end of a pentadeoxynucleotide (sequence 5′-AAAAT-3′) and further extension by a pentathymidylate. Upon cleavage/deprotection and desalting, the photocleavable oligodeoxynucleotide was irradiated at 352 nm for 2 h on (a 16W UV lamp). The irradiated solution, directly measured by Electrospray Mass Spectrometry (ES-MS), displayed two peaks corresponding to both pentadeoxynucleotides (bearing a terminal phosphate either at 5′ or 3′-end) resulting from the cleavage of both orthophenyl moieties (scheme 4).

Using the phosphoramidite 4, a photocleavable chimeric DNA/RNA was synthesized using standard phosphoramidite chemistry on a 96-well Mermade synthesizer. The oligonucleotide consisted of a dodecathymilydate followed by the bis-ortho-nitrobenzyl linker and further extended with two deoxynucleotides followed by a 19 nt long oligoribonucleotide. The chimera was prepared in the “trityl-on” mode purified by reverse-phase cartridge and analyzed by Mass Spectrometry before and after light irradiation (366 nm for 15 min. at room temperature). Two peaks were detected corresponding to the dodecathymidylate bearing a phosphate residue on its 5′-terminus and the 21 nt long DNA/RNA chimera with a 3′-phosphate residue.

We then synthesized on a 96-well Mermade synthesizer one long DNA/RNA chimera composed of two complementary strands separated by the bis-ortho-nitrobenzyl linker. Each strand was formed of a deoxynucleotide dimer on its 3′-end and a 19-nt long oligoribonucleotide. The chimera was prepared in the “trityl-on” mode, purified by reverse-phase cartridge and analyzed by Mass Spectrometry before and after light irradiation (366 nm for 15 min. at room temperature). Before irradiation we observed a unique peak corresponding to the full-length material. After irradiation, the masses corresponding to both strands were observed with a complete disappearance of starting material.

1. Process for the preparation of an oligomeric compound made up of two or more individual oligomers, in which said oligomeric compound the individual oligomers are separated by a photocleavable linker, comprising the step of photoactively cleaving said linker.

2. Process according to claim 1 wherein the individual oligomers are independently chosen from the group consisting of oligonucleotides, oligosaccharides and oligopeptides.

3. Process according to claim 1 wherein the individual oligomers are oligonucleotides.

4. Process according to claim 1 wherein the individual oligomers are oligonucleotides which are fully or partially complementary.

5. Process according to claim 1 wherein the individual oligomers are oligoribonucleotides which are fully or partially complementary.

6. Process according to claim 1 wherein the linker is stable under the deprotection conditions of each individual oligomer.

7. Process according to claim 1 wherein the linker group is cleaved by UV or visible light irradiation.

8. Process according to claim 4 wherein said oligonucleotides are two oligoribonucleotides

9. Process according to claim 1, wherein the linker is a compound of formula I, 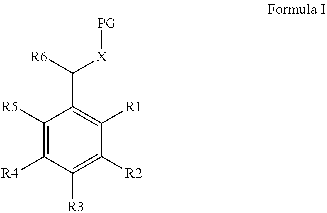
wherein;
PG is (Ar1)(Ar2)(Ar3)C—, wherein Ar1, Ar2, Ar3 are independently chosen from the group consisting of;
CH₃OC₆H₄— and C₆H₅—,
or PG is a substituted silyl group (R1′)(R2′)(R3′)Si—,
wherein R1′, R2′, R3′ is independently chosen from the group consisting of lower alkyl, aryl, aryl lower alkyl, lower alkylaryl, lower alkyloxy, or aryloxy;
X is O, N, or S;
R1, R2, R3, R4, and R5 is independently chosen from the group consisting of hydrogen, lower alkyl, aryl, aryl lower alkyl, lower alkylaryl, lower alkylhalogen, halogen, CN, COOH, C(O)O lower alkyl/aryl, CONR′R″, CHO, C(O) lower alkyl/aryl, OH, O-lower alkyl/aryl, OC(O) lower alkyl/aryl, SH, S-lower alkyl/aryl, SO₃H, SO₂O-lower alkyl/aryl, SO₂NR′R″, NH₂, N-lower alkyl/aryl, NHC(O) lower alkyl/aryl, and at least one of the substituents R1-R5 is a nitro, a nitrosyl, or a diazo group;
two or more of the substituents R1, R2, R3, R4, and R5 can form one or several rings which may be further substituted with groups defined as for R1, R2, R3, R4, or R5;
at least one of the substituents R1, R2, R3, R4, or R5 is a phosphoramidite, a phosphonate, or a phosphotriester bearing group able to form a phosphodiester or phosphorothioate linkage to the growing oligonucleotide chain or an amine, an activated carboxylic ester, an isocyanate or an isothiocyanate, able to form an amide, a urea or a thiourea linkage to the growing oligonucleotide chain;
R6 is chosen from the group consisting of hydrogen, lower alkyl, aryl, aryl lower alkyl, lower alkylaryl, lower alkylhalogen, CN, COOH, C(O)O lower alkyl/aryl, CONR′R″, CHO, C(O) lower alkyl/aryl, O-lower alkyl/aryl, OC(O) lower alkyl/aryl, S-lower alkyl/aryl, SO₃H, SO₂O-lower alkyl/aryl, SO₂NR′R″, N-lower alkyl/aryl, NHC(O) lower alkyl/aryl.

10. Process according to claim 1, wherein the linker is a compound of formula II, 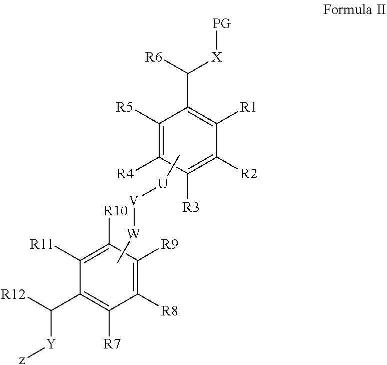
wherein,
PG is (Ar1)(Ar2)(Ar3)C—, wherein Ar1, Ar2, Ar3 are independently chosen from the group consisting of;
CH₃OC₆H₄—, C₆H₅—,
or PG is a substituted silyl group (R1′)(R2′)(R3′)Si—,
wherein R1′, R2′, R3′ is independently chosen from the group consisting of lower alkyl, aryl, aryl lower alkyl, lower alkylaryl, lower alkyloxy, or aryloxy;
X is O, N, or S;
R1, R2, R3, R4, and R5 is independently chosen from the group consisting of hydrogen, lower alkyl, aryl, aryl lower alkyl, lower alkylaryl, lower alkylhalogen, halogen, CN, COOH, C(O)O lower alkyl/aryl, CONR′R″, CHO, C(O) lower alkyl/aryl, OH, O-lower alkyl/aryl, OC(O) lower alkyl/aryl, SH, S-lower alkyl/aryl, SO₃H, SO₂O-lower alkyl/aryl, SO₂NR′R″, NH₂, N-lower alkyl/aryl, NHC(O) lower alkyl/aryl, and at least one of the substituents R1-R5 is a nitro, a nitrosyl, or a diazo group;
two or more of the substituents R1, R2, R3, R4, and R5 can form one or several rings which may be further substituted with groups defined as for R1, R2, R3, R4, or R5;
R6 is chosen from the group consisting of hydrogen, lower alkyl, aryl, aryl lower alkyl, lower alkylaryl, lower alkylhalogen, CN, COOH, C(O)O lower alkyl/aryl, CONR′R″, CHO, C(O) lower alkyl/aryl, O-lower alkyl/aryl, OC(O) lower alkyl/aryl, S-lower alkyl/aryl, SO₃H, SO₂O-lower alkyl/aryl, SO₂NR′R″, N-lower alkyl/aryl, NHC(O) lower alkyl/aryl;
U, V, W are forming a chain which replaces one of the substituents R1-R5 on one end and one of the substituents R7-R11 on the other end;
U, V, W can independently be absent, or be an alkylene (—R—), cycloalkylene (—R—), or arylene (—Ar—) group, —O—, —S—, —NR′—, —C(O)—, —C(O)O—, —C(O)NR′—, —OC(O)O—, —OC(O)NR′—, —NR′C(O)NR″—, —OC(S)NR′—, —NR′C(S)NR″—, —S(O)—, —S(O₂)—, —S(O₂)NR′—, —OP(O₂)O—, and may contain a label or fluorophore or a group which serves to improve the pharmacological profile of the oligonucleotide.
R7, R8, R9, R10, and R11 are independently chosen form the group consisting of,
hydrogen, lower alkyl, aryl, aryl lower alkyl, lower alkylaryl, lower alkylhalogen, halogen, CN, COOH, C(O)O lower alkyl/aryl, CONR′R″, CHO, C(O) lower alkyl/aryl, OH, O-lower alkyl/aryl, OC(O) lower alkyl/aryl, SH, S-lower alkyl/aryl, SO₃H, SO₂O lower alkyl/aryl, SO₂NR′R″, NH₂, N-lower alkyl/aryl, NHC(O) lower alkyl/aryl and at least one of the substituents R7-R11 is a nitro, a nitrosyl, or a diazo group;
R12 is chosen from the group consisting of hydrogen, lower alkyl, aryl, aryl lower alkyl, lower alkylaryl, lower alkylhalogen, CN, COOH, C(O)O lower alkyl/aryl, CONR′R″, CHO, C(O) lower alkyl/aryl, O-lower alkyl/aryl, OC(O) lower alkyl/aryl, S-lower alkyl/aryl, SO₃H, SO₂O-lower alkyl/aryl, SO₂NR′R″, N-lower alkyl/aryl, NHC(O) lower alkyl/aryl;
Y is O, N, or S;
Z is a phosphoramidite, a phosphonate, or a phosphotriester group able to form a phosphodiester or phosphorothioate linkage to the growing oligonucleotide chain or an amine, an activated carboxylic ester, an isocyanate or an isothiocyanate which is able to form an amide, a urea or a thiourea linkage to the growing oligonucleotide chain.

11. Process according to claim 1, wherein the linker is a compound according to formula I, 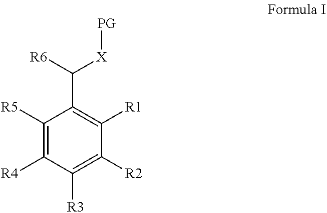
wherein;
PG is dimethoxytriphenylmethyl;
X is O;
R1 is a nitro group;
R3 is —CH₂—O—P(N[iPr]₂)—O—CH₂—CH₂—CN);
R2, R4, R5, and R6 are hydrogen;

12. Process according to claim 1, wherein the linker is a compound of formula II 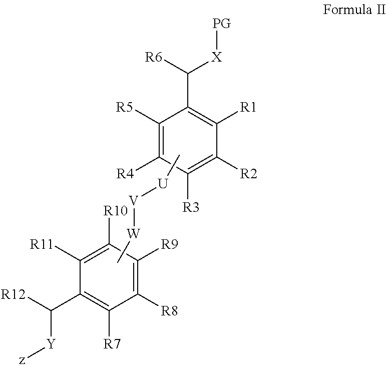
wherein;
PG is dimethoxytriphenylmethyl;
X and Y are O;
R1 and R7 are nitro groups;
R2, R4, R5, R6, R8, R10, R11, and R12 are hydrogen;
U is oxygen and replaces R3;
V is —CH₂—CH₂—CH₂—;
W is oxygen and replaces R9;
Z is —P(N[iPr]₂)—O—CH₂—CH₂—CN).

13. A compound of formula I, 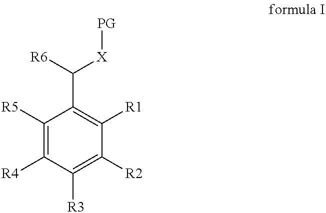
wherein;
PG is (Ar1)(Ar2)(Ar3)C—, wherein Ar1, Ar2, Ar3 are independently chosen from the group consisting of;
CH₃OC₆H₄— and C₆H₅—,
or PG is a substituted silyl group (R1′)(R2′)(R3′)Si—,
wherein R1′, R2′, R3′ is independently chosen from the group consisting of lower alkyl, aryl, aryl lower alkyl, lower alkylaryl, lower alkyloxy, and aryloxy;
X is O, N, or S;
R1, R2, R3, R4, and R5 is independently chosen from the group consisting of hydrogen, lower alkyl, aryl, aryl lower alkyl, lower alkylaryl, lower alkylhalogen, halogen, CN, COOH, C(O)O lower alkyl/aryl, CONR′R″, CHO, C(O) lower alkyl/aryl, OH, O-lower alkyl/aryl, OC(O) lower alkyl/aryl, SH, S-lower alkyl/aryl, SO₃H, SO₂O-lower alkyl/aryl, SO₂NR′R″, NH₂, N-lower alkyl/aryl, and NHC(O) lower alkyl/aryl; and at least one of the substituents R1-R5 is a nitro, a nitrosyl, or a diazo group;
two or more of the substituents R1, R2, R3, R4, and R5 can form one or several rings which may be further substituted with groups defined as for R1, R2, R3, R4, or R5;
at least one of the substituents R1, R2, R3, R4, or R5 is a phosphoramidite, a phosphonate, or a phosphotriester bearing group able to form a phosphodiester or phosphorothioate linkage to the growing oligonucleotide chain or an amine, an activated carboxylic ester, an isocyanate or an isothiocyanate, able to form an amide, a urea or a thiourea linkage to the growing oligonucleotide chain;
R6 is chosen from the group consisting of hydrogen, lower alkyl, aryl, aryl lower alkyl, lower alkylaryl, lower alkylhalogen, CN, COOH, C(O)O lower alkyl/aryl, CONR′R″, CHO, C(O) lower alkyl/aryl, O-lower alkyl/aryl, OC(O) lower alkyl/aryl, S-lower alkyl/aryl, SO₃H, SO₂O-lower alkyl/aryl, SO₂NR′R″, N-lower alkyl/aryl, and NHC(O) lower alkyl/aryl.

14. A compound according to claim 13, wherein;
PG is dimethoxytriphenylmethyl;
X is O;
R1 is a nitro group;
R3 is —CH₂—O—P(N[iPr]₂)—O—CH₂—CH₂—CN);
R2, R4, R5, and R6 are hydrogen;

15. A compound of formula II 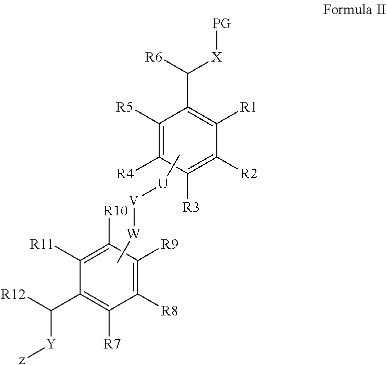
wherein,
PG is (Ar1)(Ar2)(Ar3)C—, wherein Ar1, Ar2, Ar3 are independently chosen from the group consisting of;
CH₃OC₆H₄—, C₆H₅—,
or PG is a substituted silyl group (R1′)(R2′)(R3′)Si—,
wherein R1, R2′, R3′ is independently chosen from the group consisting of lower alkyl, aryl, aryl lower alkyl, lower alkylaryl, lower alkyloxy, or aryloxy;
X is O, N, or S;
R1, R2, R3, R4, and R5 is independently chosen from the group consisting of hydrogen, lower alkyl, aryl, aryl lower alkyl, lower alkylaryl, lower alkylhalogen, halogen, CN, COOH, C(O)O lower alkyl/aryl, CONR′R″, CHO, C(O) lower alkyl/aryl, OH, O-lower alkyl/aryl, OC(O) lower alkyl/aryl, SH, S-lower alkyl/aryl, SO₃H, SO₂O-lower alkyl/aryl, SO₂NR′R″, NH₂, N-lower alkyl/aryl, and NHC(O) lower alkyl/aryl, and at least one of the substituents R1-R5 is a nitro, a nitrosyl, or a diazo group;
two or more of the substituents R1, R2, R3, R4, and R5 can form one or several rings which may be further substituted with groups defined as for R1, R2, R3, R4, or R5;
R6 is chosen from the group consisting of hydrogen, lower alkyl, aryl, aryl lower alkyl, lower alkylaryl, lower alkylhalogen, CN, COOH, C(O)O lower alkyl/aryl, CONR′R″, CHO, C(O) lower alkyl/aryl, O-lower alkyl/aryl, OC(O) lower alkyl/aryl, S-lower alkyl/aryl, SO₃H, SO₂O-lower alkyl/aryl, SO₂NR′R″, N-lower alkyl/aryl, and NHC(O) lower alkyl/aryl;
U, V, W are forming a chain which replaces one of the substituents R1-R5 on one end and one of the substituents R7-R11 on the other end;
U, V, W can independently be absent, or be an alkylene (—R—), cycloalkylene (—R—), or arylene (—Ar—) group, —O—, —S—, —NR′—, —C(O)—, —C(O)O—, —C(O)NR′—, —OC(O)O—, —OC(O)NR′—, —NR′C(O)NR″—, —OC(S)NR′—, —NR′C(S)NR″—, —S(O)—, —S(O₂)—, —S(O₂)NR′—, —OP(O₂)O—, and may contain a label or fluorophore or a group which serves to improve the pharmacological profile of the oligonucleotide.
R7, R8, R9, R10, and R11 are independently chosen form the group consisting of,
hydrogen, lower alkyl, aryl, aryl lower alkyl, lower alkylaryl, lower alkylhalogen, halogen, CN, COOH, C(O)O lower alkyl/aryl, CONR′R″, CHO, C(O) lower alkyl/aryl, OH, O-lower alkyl/aryl, OC(O) lower alkyl/aryl, SH, S-lower alkyl/aryl, SO₃H, SO₂O lower alkyl/aryl, SO₂NR′R″, NH₂, N-lower alkyl/aryl, NHC(O) lower alkyl/aryl and at least one of the substituents R7-R11 is a nitro, a nitrosyl, or a diazo group;
R12 is chosen from the group consisting of hydrogen, lower alkyl, aryl, aryl lower alkyl, lower alkylaryl, lower alkylhalogen, CN, COOH, C(O)O lower alkyl/aryl, CONR′R″, CHO, C(O) lower alkyl/aryl, O-lower alkyl/aryl, OC(O) lower alkyl/aryl, S-lower alkyl/aryl, SO₃H, SO₂O-lower alkyl/aryl, SO₂NR′R″, N-lower alkyl/aryl, NHC(O) lower alkyl/aryl;
Y is O, N, or S;
Z is a phosphoramidite, a phosphonate, or a phosphotriester group able to form a phosphodiester or phosphorothioate linkage to the growing oligonucleotide chain or an amine, an activated carboxylic ester, an isocyanate or an isothiocyanate which is able to form an amide, a urea or a thiourea linkage to the growing oligonucleotide chain.

16. A compound according to claim 15, wherein;
PG is dimethoxytriphenylmethyl;
X is O;
R1 is a nitro group;
R3 is —CH₂—O—P(N[iPr]₂)—O—CH₂CH₂—CN);
R2, R4, R5, and R6 are hydrogen;
